(19) United States
(12) Patent Application Publication
(10) Pub. No.: US 2002/0164336 A1
(43) Pub. Date: Nov. 7, 2002

(54) Combination anti-selectin and immunosuppressant therapy

(76) Inventors: Paul C. Harrison, Naugatuck, CT (US); Jeffrey B. Madwed, Danbury, CT (US); Robert Rothlein, Danbury, CT (US); Takashi K. Kishimoto, New Fairfield, CT (US)

Correspondence Address: BOEHRINGER INGELHEIM CORPORATION, 900 RIDGEBURY ROAD, P O BOX 368, RIDGEFIELD, CT, 06877, US

(21) Appl. No.: 10/140,849

(22) Filed: May 8, 2002

Related U.S. Application Data
(63) Continuation of application No. 09/250,777, filed on Feb. 12, 1999, now abandoned.
(60) Provisional application No. 60/076,035, filed on Feb. 26, 1998.

(51) Int. Cl. A61K 39/395; A61K 38/13

(52) U.S. Cl. 424/146.1; 514/9

(57) ABSTRACT

A combination therapy of administering to a patient at least one selectin inhibitor and at least one immunosuppressant is employed in methods for modulating the patient's immune response, and in treatment regimens for organ and tissue transplant rejections and various inflammatory disorders. In some embodiments, the selectin inhibitor is an antibody to L-selectin or P-selectin such as a Dreg series of monoclonal antibody, or a functional fragment thereof, or SLe^{x} or a SLe^{x} derivative or mimetic, and the immunosuppressant is cyclosporin A.

### TECHNICAL FIELD OF THE INVENTION

This invention relates primarily to the use of selectin inhibitors in combination with other immunosuppressants for the prevention of acute allograft rejection and in the treatment of autoimmune diseases and other pathological disorders involving inflammation.

### BACKGROUND OF THE INVENTION

Immunosuppressive agents are employed to reduce the body's natural immunity in patients who receive organ, e.g., kidney, liver, and heart transplants. Since these drugs interfere with the body's immune system and thus compromise infection resistance, they must be carefully administered and monitored. Moreover, many may cause serious side effects. Cyclosporin A (sometimes called cyclosporine or ciclosporin and marketed under the brand name Sandimmune®), for example, may cause high blood pressure and kidney and liver problems, as well as tremors and gum hyperplasia (Complete Drug Reference, 1993 ed., U.S. Pharmacoeia, Consumer's Union, Yonkers, N.Y., pp 465-468). Cyclosporin A and other immunosuppressives employed post-transplantation may cause a variety of other symptoms, including nausea, fatigue, and hair loss, and are themselves carcinogens in some cases (see, for example, Wyngaarden, J. B., et al., eds., Cecil's Textbook of Medicine, W. B. Saunders, Philadelphia, 1992, Table 158-4, p. 1031).

Improved immunosuppressant therapies and related treatments of immune system disorders such as autoimmune disease have been the subject of investigation for many years. It is now known that leukocyte recruitment into inflammatory sites is a multi-step process which involves an initial transient contact of the cells with the endothelium, called rolling, followed by adhesion and transmigration (Ley, K., et al., 1995, J. Exp. Med. 181: 669-675). Leukocyte rolling is characterized by rapid formation and subsequent breakage of bonds formed by selectins (ibid.) Selectins, which include L-selectin, E-selectin, and P-selectin, are calcium-dependent 138 mammalian adhesion molecules that share a common structural motif: an N-terminal C-type lectin domain, an epidermal growth factor-homologous domain, a variable number of short consensus repeats found in many complement regulatory proteins (e.g., factor H), a transmembrane domain, and a C-terminal cytoplasmic domain (Kishimoto, T. K., and Rothlein, R., 1994, Adv. Pharm. 25:117-169). The selectins have distinct tissue distributions: L-selectin is expressed on circulating granulocytes, monocytes, and most lymphocytes; E-selectin is induced on cytokine-treated endothelial cells; and P-selectin is expressed on the surface of platelets and endothelial cells shortly after stimulation and is further induced by exposure of endothelial cells to cytokines (ibid).

Selectin function and the nature of selectin ligands have been a recent focus of research in many laboratories. Though there are ambiguities due to the subtle complexities of the inflammatory process and the structure-function and receptor-ligand interactions related to the selecting, several lines of evidence have shed light on the physiological roles of these adhesion molecules and others, such as the integrins and ICAMs (intercellular adhesion molecules). It has been found, for example, that in mice rendered genetically deficient for either P-selectin or L-selectin, granulocyte emigration into an experimentally inflamed peritoneum is significantly attenuated (Ley, et at., cited above), and that simultaneously blocking L- and P-selectin with antibodies completely inhibits neutrophil migration into the murine peritoneum (Bosse, R., and Vestweber, D., 1994, Eur. J. Immunol. 24: 3019-3024). Leukocyte emigration into inflammatory sites is also impaired in animals treated with several selectin-blocking antibodies. These findings indicate that selectin-mediated rolling is an important determinant of the inflammatory, response and precedes inflammatory cell emigration (Ley, et al., cited above).

On the basis of these results, several investigators have further experimented and speculated that various adhesive molecules (including the selectins) might have therapeutic application in a variety of inflammatory and immunological diseases and/or pathological conditions. Though anti-P-selectin antibodies were shown not to interfere with platelet-graft interactions in one baboon study, leukocyte-platelet interactions were blocked, as well as deposition of fibrin within an experimental thrombus (Palabrica, T., et al., 1992, Nature 239:848-851). P-selectin was found to be co-expressed with ICAM-1 in the endothelium overlying atheroschlerotic plaques in human arterial sections obtained after reconstructive and postmortem surgery (Johnson-Tidey, R. R., et al., 1994, Am. J. Path. 144: 952-961). Pretreatment with anti-ICAM-1, but not anti-E-selectin, inhibited the eosinophil influx and onset of airway inflammation and hyperresponsiveness after antigen inhalation in a primate asthma model, but anti-ICAM-1 did not reverse an existing condition of airway inflammation or hyperresponsiveness, whereas late-phase airway obstruction associated with acute airway inflammation after a single antigen inhalation (not affected by anti-ICAM-1) was significantly attenuated by anti-E-selectin antibodies (reviewed by Kishimoto and Rothlein, cited above.). E-selectin and P-selectin have also been shown to have some involvement in a rat model of immune complex injury in the lung (ibid.). P-selectin and tumor necrosis factor-α inhibition reduced thrombosis inflammation in rats (Wakefield, T. W., et al., 1996, J. Surg. Res. 64: 26-31). Monoclonal antibodies to E-selectin and L-selectin and their ligands increased hepatic blood flow upon reperfusion following cold ischemia in murine liver transplantation studies, though recovery of microcirculation was not perfect and there was no improvement in bile production (Hamamoto, I., et al., 1996, Transpl. Int. 9: 454-460). Lymphocyte adhesion to endothelium after rat heart transplants was found to be significantly decreased by treating the lymphocytes with anti-L-selectin antibody (Turunen, J.P., et. al., 1995, J. Exp. Med. 182: 1133-1142). Delayed rejection of allografts in L-selectin-deficient mice has also been reported (Tang, M. L. K., et al., 1997, J. Immun. 5191-5199). Even though the results in some of the studies are inconclusive, selectin inhibitors have been suggested as possible therapeutic agents for use in modulating the course of inflammation, cancer, or other diseases involving unwanted cell-cell adhesion (see, for example, U.S. Pat. No. 5,440,015 to Macher and Briggs).

### SUMMARY OF THE INVENTION

It is an objective of the invention to provide a therapeutic regimen that overcomes some of the disadvantages of currently employed immunosuppressant therapies.

It is another objective of the invention to provide improved alternative therapies and regimens for the treatment of tissue or organ rejection and various pathological conditions involving inflammation.

These and other objectives are accomplished by the present invention, which provides methods for modulating the immune response in a patient and treatments for tissue and organ rejections and various pathological disorders involving inflammation, including autoimmune diseases, by administration to the patient of an effective amount of at least one selectin inhibitor in combination with at least one immunosuppressant to the patient. Selectins include E-selectin, L-selectin, P-selectin, and mixtures thereof; L-selectin or P-selectin are inhibited in many embodiments. Inhibitors include antibodies to the selectins, functional fragments thereof, and other compounds that inhibit selectin function such as SLe^{x} (a myeloid-specific sialylated fucosylated carbohydrate moiety denominated sialyl Lewis X, herein referred to as SLe^{x}, summarized in Kishimoto and Rothlein, cited above), and/or SLe^{x} derivatives and mimetics. Immunosuppressants used in the combination treatments include, but are not limited to, cyclosporin A, rapamycin and FK-506. The invention correspondingly provides improvements in immunosuppressant therapies, and pharmaceutical compositions and regimens employing a combination of at least one selectin inhibitor and at least one immunosuppressant at dosage levels that are efficacious and yet mimimize toxic side effects.

### DETAILED DESCRIPTION OF THE INVENTION

This invention is based upon the finding that selectin inhibitors that alter leukocyte rolling can be used in combination with an immunosuppressant to provide a therapy that is more efficacious than either the inhibitor or the immunosuppressant alone, particularly for the treatment of acute allograft rejection and various other inflammatory disorders. As mentioned above, since many current immunosuppressant therapies are toxic, the invention thus provides a way of decreasing the immunosuppressant dose and consequent ill effects, while simultaneously providing an efficacious treatment.

The invention is directed to treatments for organ and tissue transplant rejection and diseases and pathological conditions involving inflammation. These encompass chronic inflammatory diseases including, but not limited to, rheumatoid arthritis, multiple sclerosis, Guillain-Barre syndrome, Crohn's disease, ulcerative colitis, psoriasis, lupus erythematosus, insulin-dependent diabetes mellitus, psoriasis, psoriatic arthritis, sarcoidosis, hypersensitivity pneumonitis, ankylosing spondylitis and related spoldyloahthropathies, Reiter's syndrome, systemic sclerosis, and the like, as well as a number of diseases of autoimmunity including toxic shock syndrome, osteoarthritis, and inflammatory bowel disease. As used herein, the term “inflammation” is used to refer to reactions of both the specific and non-specific defense systems and thus includes inflammatory responses to bee stings, bacterial infections, frost-bite injury, surgical wound healing, and so forth.

In the practice of the invention, the immune response of a patient is modulated and the patient, treated for transplant rejection or inflammatory disorders by administering to the patient a combination therapy comprising at least one selectin inhibitor and at least one immunosuppressant. Selectins include E-selectin, L-selectin, P-selectin, and mixtures of any of these. As summarized above, L-selectin or P-selectin are inhibited in many embodiments. The invention has both medical and veterinary applications, and so, as used herein, a “patient” may be a human being or an animal.

Selectin inhibitors include, but are not limited to, polyclonal, monoclonal, and fusion phage antibodies, functional fragments thereof, other compounds that inhibit selectin function, and mixtures of any of these. By “antibody” is meant an immunoglobulin having a specific amino acid sequence by virtue of which it interacts with antigen induced in cells of the lymphoid series, Fab fragments that function similarly, and the like. Numerous selectin antibody inhibitors have been described in human and animal models including, but not limited to, antibodies denoted as the Dreg series of monoclonal antibodies (described by Kishimoto, T. K, et al., 1990, P.N.A.S. 87: 2244-2248), EL-246 (Jutila, M., et al., 1992, J. Exp. Med. 175: 1565-1573) and mixtures of these; HRL-1 and HRL-2 (employed, for example, by Turunen, J.P., et al., 1994, Eur. J. Immunol. 24: 1130-1136); mAb 10E9.6, RB40.34, and mAb 21KC10 (Bosse and Vestweber, and Ley, et al., cited above); PB1.3 and P7 (Wakefield, et al., cited above); and products obtained commercially such as L-selectin and E-selectin monoclonal antibodies that can be purchased from Seikagaku. Use of an anti-L-selectin antibody denoted HRL-3 is illustrated hereafter. Some of the previously described antibodies are to rat, hamster, guinea pig, and mouse selecting. As used herein, the term “antibody” includes humanized counterparts of these polypeptides, and functional fragments thereof.

Other compounds, including small synthetic chemical compounds such as SLe^{x}, SLe^{x} derivatives and mimetics and mixtures of these with each other and with SLe^{x}, and the like may also be used as selectin inhibitors, alone, or in combination with each other, or with antibodies or functional antibody fragments. Any chemical or biochemical compound that inhibits selectin function may be employed in the practice of the invention. These include, but are not limited to, large molecular entities such as sulfatide fucotides, ppmE₁, dextran sulfate and smaller molecular entities described above and/or soluble natural ligands. As used herein, a “selectin inhibitor” includes selectin antagonists and other compounds such as antibodies that bind to selectin itself as well as compounds that inhibit selectin function by binding to selectin receptors or ligands. Assays for selectin inhibitors have been described, for example, by Rosen, et al., in U.S. Pat. No. 5,318,890, and references cited therein.

Treatment and therapy regimens according to the invention further include administration of an immunosuppressant to the patient. Immunosuppressants include, but are not limited to, the cyclosporins (particularly cyclosporin A), rapamycin (Sirolimus™), FK-506 (Tacrolimus™), and the like and mixtures thereof. Cyclosporin A is employed in one preferred embodiment. Preferred embodiments employ an immunosuppressive dose that is insufficient to provide an immunosuppressant effect in the patient in the absence of a concomitantly administered selectin inhibitor. As summarized above, it is an advantage of the invention that use of a selectin inhibitor in conjunction with a toxic immunosuppressant enhances the effectiveness of the treatment, thereby lowering the dose of immunosuppressant required.

The immunosuppressant and selectin inhibitor combination of this invention may be administered in any conventional dosage form in any conventional manner. Such methods of treatment, including their dosage levels and other requirements, may be selected by those of ordinary skill in the art from available methods and techniques. For example, in some embodiments, the components may be combined with a pharmaceutically acceptable carrier or adjuvant for administration to a patient in need of such treatment in a pharmaceutically acceptable manner and in an amount effective to treat inflammation and diseases and pathological conditions involving inflammation (including lessening the severity of symptoms in a chronic inflammatory disease). The invention thus provides pharmaceutical compositions incorporating both components used in the methods described herein.

In alternate embodiments, the components are administered separately, either serially or in parallel. Separate dosing allows for greater flexibility in the dosing regime. In either combined, serial, or parallel dosings, the selectin inhibitor and immunosuppressant may be administered alone or in combination with adjuvants that enhance stability of the ingredients, facilitate administration of pharmaceutic compositions containing them in certain embodiments, provide increased dissolution or dispersion, increase activity, provide adjunct therapy, and the like, including other active ingredients that may further lower toxic dosage levels of the immunosuppressants.

According to this invention, the components of the therapy and pharmaceutical compositions containing them may be administered to a patient in any conventional manner and in any pharmaceutically acceptable dosage form, including, but not limited to, intravenously, intramuscularly, subcutaneously, intrasynovially, by infusion, sublingually, transdermally, orally, topically or by inhalation. Preferred modes of administration are oral and intravenous.

As mentioned above, dosage forms of the components of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmtaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. As the skilled worker will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

This invention thus provides a novel therapeutic method for treating allograft rejections and other inflammatory disorders. It also provides an improvement in current immunosuppressant therapy by providing efficacious treatments employing immunosuppressants at lower dosgae levels that minimize side effects.

The following examples are presented to further illustrate and explain the present invention and should not be taken as limiting in any regard.

### EXAMPLES

This example illustrates a solid organ allograft transplantation using a rat heterotopic cardiac model. After transplantation, the control group was administered no selectin inhibitors or immunosuppressants. A second group was given cyclosporin A (100 mg/ml Sandimmune® oral solution obtained from Sandoz Pharmaceuticals, East Hanover, N.J., denoted below as CsA; 1.5 mg/kg) as an immunosuppressant after transplantation. A third group was given an anti-L-selectin monoclonal antibody (MAb HRL-3, Tamashani, et al., Eur. J. Immunol. 1993, 23: 2184-2185; 3 mg/kg), and a fourth group was given a combination post-transplantation therapy of both cyclosporin A and anti-L-selectin MAb HRL-3). Antibody was administered to the recipient twice pre-transplantation as a loading dose, and then pest-transplantation for 10 days.

Male ACI rats (RT-1^{av1}) weighing 200 to 300 g were obtained from Harlan Sprague Dawley Co., Indianapolis, Ind. and were used as donors. Male Lewis rats (RT-1¹) weighing 280 to 400 g were obtained from Charles River Laboratories, Wilmington, Mass. were used as recipients. These two rat strains were-mismatched at the major histocompatibility loci (RT-1) and have been previously reported to be strongly immunogenic. Donor hearts were anastomosed to the recipient infrarenal vessels using a modification of a procedure described by Ono and Lindsey (1969, J. Thoracic Cardiovasc. Surg. 57: 225-229). Briefly, donor rats were anesthetized using a combination of atropine sulfate (0.05 mg/kg, SC), ketamine hydrochloride (80 mg/kg, IP) and xylazine hydrochloride (10 mg/kg, IP). The chest was opened, the inferior and superior vena cava along with the pulmonary veins were ligated, and the heart was then excised by ligating the pulmonary artery and aorta. The heart was immediately placed in cold heparinized Lactated Ringers solution. Recipient rats were anesthetized with ketamine (60 mg/kg, IM) and sodium pentobarbital (20 mg/kg, IP; Nembutal® Abbott, North Chicago, Ill.). The aorta of the donor heart was anastomosed to the abdominal aorta of the recipient, while the pulmonary artery of the donor heart was anastomosed to the abdominal inferior vena cava of the recipient. Upon reperfusion, the donor hearts generally became distended, pink, and began beating within 20 to 30 seconds. Graft survival was monitored by daily palpitation of the beating heart through the abdominal wall. Rejection was defined as the complete cessation of ventricular contractions and recorded as days to rejection with day 0 being the day of the transplantation.

Using this procedure, the following results were obtained:

| Groups | Mean Rejection Time | Individual Rejection Times |
|---|---|---|
| None | 8.8 ± 0.6 | 7, 10, 10, 10, 9, 7 |
| CsA-1.5 | 8.5 ± 0.3 | 9, 8, 8, 8, 8, 10 |
| HRL-3 | 12.3 ± 1.8 | 7, 9, 10, 13, 17, 18 |
| CsA-1.5 + HRL-3 | 20.0 ± 2.2 (P < 0.05) | 13, 17, 16, 22, 21, 20, 31 |

The results clearly show the significant superiority of combination therapy. Anti-L-selectin antibody alone did not prolong allograft survival. However, in combination with a non-therapeutic dose of cyclosporin A, a significant extension in graft survival was demonstrated.

All papers and patents cited herein are hereby fully incorporated by reference.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method for modulating the immune response in a patient comprising administering to the patient an effective amount of a combination of at least one selectin inhibitor and at least one immunosuppressant.

2. A method according to claim 1 wherein the selectin inhibitor is an antibody to a selectin, or a fragment thereof, which inhibits selectin function.

3. A method according to claim 2 wherein the antibody is a Dreg series monoclonal, or a fragment or variant thereof, or mixtures of any of these.

4. A method according to claim 1 wherein the selectin inhibitor is a compound that is an antagonist of selectin function.

5. A method according to claim 4 wherein the compound is SLe^{x}, a SLe^{x} derivative, a SLe^{x} mimetic, or mixtures thereof.

6. A method according to claim 1 wherein the selectin is selected from the group consisting of E-selectin, L-selectin, P-selectin, and mixtures thereof.

7. A method according to claim 6 wherein the selectin is L-selectin or P-selectin.

8. A method according to claim 1 wherein the immunosuppressant is cyclosporin A.

9. A method according to claim 1 comprising a treatment for organ or tissue transplant rejection.

10. A method according to claim 1 comprising a treatment for an inflammatory disorder selected from the group consisting of rheumatoid arthritis, multiple sclerosis, Guillain-Barre syndrome, Crohn's disease, ulcerative colitis, psoriasis, lupus erythematosus, insulin-dependent diabetes mellitus, psoriatic arthritis, sarcoidosis, hypersensivity pneumonitis, ankylosing spondylitis and related spoldyloarthropathies, Reiter's syndrome and systemic sclerosis.

11. An improvement in immunosupressant therapy comprising administering to a patient in need thereof an effective amount of at least one immunosuppressant in combination with at least one selectin inhibitor.

12. An improvement according to claim 11 wherein the immunosuppressant is administered to the patient in an amount insufficient to provide an immunosuppressant effect in the patient in the absence of the selectin inhibitor.

13. An improvement according to claim 11 wherein the selectin inhibitor is selected from the group consisting of an antibody to E-selectin, an antibody to L-selectin, an antibody to P-selectin, functional fragments, and mixtures thereof.

14. An improvement according to claim 11 wherein the selectin inhibitor is a compound that inhibits selectin function.

15. An improvement according to claim 11 wherein the selectin is L-selectin or P-selectin, and the immunosuppressant is cyclosporin A.

16. A pharmaceutical composition comprising at least one immunosuppressant and at least one selectin inhibitor.

17. A composition according to claim 16 wherein the selectin is L-selectin or P-selectin.

18. A composition according to claim 16 wherein the inhibitor is selected from the group consisting of an antibody to the selectin, a functional fragment thereof, SLe^{x}, SLe^{x} derivatives, SLe^{x} mimetics, and mixtures thereof.

19. A composition according to claim 16 wherein the immunosuppressant is cyclosporin A.

20. Use of a selectin inhibitor with an immunosupressant to modulate the immune response of a patient.
